(19) United States
(12) Patent Application Publication
(10) Pub. No.: US 2004/0106954 A1
(43) Pub. Date: Jun. 3, 2004

(54) Treatment of congestive heart failure

(76) Inventors: Todd K. Whitehurst, Santa Clarita, CA (US); James P. McGivern, Stevenson Ranch, CA (US); Kelly H. McClure, Simi Valley, CA (US); James R. Thacker, Eureka, MT (US)

Correspondence Address: ADVANCED BIONICS CORPORATION, 12740 SAN FERNANDO ROAD, SYLMAR, CA, 91342, US

(21) Appl. No.: 10/713,512

(22) Filed: Nov. 14, 2003

Related U.S. Application Data
(60) Provisional application No. 60/426,972, filed on Nov. 15, 2002.

(51) Int. Cl. A61N 1/362

(52) U.S. Cl. 607/3

(57) ABSTRACT

Treatment of congestive heart failure (CHF) includes implantation of the discharge portion(s) of a catheter and, optionally, electrode(s) on a lead, near the tissue(s) to be stimulated. Stimulation pulses, i.e., drug infusion pulses and optional electrical pulses, are supplied by a stimulator implanted remotely, and through the catheter or lead, which is tunneled subcutaneously between the stimulator and stimulation site. Stimulation sites include the coronary arteries, the aorta, the left ventricle, the left atrium, and/or the pulmonary veins, among other locations. Disclosed treatments include drugs used for acute treatment of CHF, for chronic treatment of CHF, and drugs to reverse CHF.

The present application claims the benefit of U.S. Provisional Patent Application Serial No. 60/426,972, filed Nov. 15, 2002, which application is incorporated herein by reference in its entirety.

### FIELD OF THE INVENTION

The present invention generally relates to implantable drug delivery and electrical stimulation systems and methods, and more particularly relates to utilizing one or more such implantable devices for treating congestive heart failure.

### BACKGROUND OF THE INVENTION

Congestive heart failure is traditionally defined as the pathophysiological state in which the heart is unable to pump blood at a rate commensurate with the body's metabolic requirements. This reduction in myocardial function is most commonly caused by ischemic injury produced by obstructive congestive heart failure, but is also a consequence of primary cardiomyopathy and anatomic lesions of the cardiac valves and pericardium.

The National Heart, Lung and Blood Institute estimates that more than two million Americans are afflicted with heart failure, with 400,000 new cases and 900,000 hospitalizations occurring each year. There are between 200,000 to 400,000 deaths annually attributed to heart failure. Surprisingly, mortality related to heart failure has increased since 1968, despite the overall decline in deaths related to cardiovascular disease. In addition to the cost of human life, heart failure poses a tremendous financial burden on the health care system. In 1990, it was the most common discharge diagnosis in persons over age 65 and accounted for annual expenditures exceeding $4.7 billion.

The most recent analysis of 34 years of follow-up in the Framingham Study found that advanced age was an important determinant of risk of heart failure. The prevalence of heart failure was about 1% for those aged 50 to 59 years and rose progressively with age to affect 10% of persons in their 80s. The annual incidence also increased with age, from about 0.2% in persons 45 to 54 years to 5.4% in men 85 to 95 years. In the Framingham study, 37% of men and 33% of women died within two years of their diagnosis in cardiac failure, and after six years, mortality rates increased 82% and 67%, respectively. This represents a death rate from four to eight times higher than that in the general population at the same age.

Diagnosing heart failure is a relatively straightforward process, although the heart itself produces no clinical symptoms when it fails as a pump. Symptoms are found instead in derangements of the lungs, kidneys, liver, and other organs. The New York Heart Association (NYHA) functional classification of heart failure is a standard method of assessing clinical status:

Class I: No limitation of physical activity; no dyspnea, fatigue or palpitations with ordinary physical activity.

Class II: Slight limitation of activity; patients have fatigue, palpitations and dyspnea with ordinary physical activity, but are comfortable at rest.

Class III: Marked limitation of activity; less than ordinary physical activity results in symptoms, but patients are comfortable at rest.

Class IV: Symptoms are present at rest and they are exacerbated by any physical exertion.

More advanced heart failure, as determined by NYHA functional class, is associated with decreased survival. However, patients become symptomatic only after marked deterioration of myocardial function, so these classes are an insensitive diagnostic tool.

Major clinical symptoms of left-heart failure are: exertional dyspnea, orthopnea, paroxysmal nocturnal dyspnea, dyspnea at rest, exercise intolerance, weakness, fatigue, nocturia, and mental confusion. Major clinical symptoms of right-heart failure are: systemic venous congestion including dependent edema, right upper quadrant pain due to stretching of the hepatic capsule from liver engorgement, anorexia, nausea, bloating due to congestion of the mesentery and liver, and fatigue. Pulmonary symptoms are unlikely unless left-heart failure is also present.

Heart failure represents an enormous clinical challenge in need of effective therapeutic approaches.

### BRIEF SUMMARY OF THE INVENTION

The invention disclosed and claimed herein provides treatment for congestive heart failure (CHF) and relieving its symptoms using one or more drugs by means of a stimulator comprising an implantable pump(s) and catheter(s) and an optional implantable signal generator(s) for additionally delivering electrical stimulation. One or more catheters, and possibly also electrodes carried on a catheter or lead, are surgically implanted in the cardiovascular system supplying blood to the heart to infuse the drugs.

The present invention overcomes the shortfalls of all prior art treatment devices to achieve unprecedented levels of CHF treatment by combining administration of acute CHF medications (e.g., nitroprusside) and chronic CHF medications (e.g., an ACE inhibitor) with agents that reverse CHF, such as agents that improve myocyte calcium handling (e.g., an adenovirus carrying a SERCA2a gene). Additionally, the present invention optionally combines electrical stimulation with delivery of one or more of these drugs for acute (on-demand) and traditional chronic (basal or periodic bolus) treatment of CHF.

The stimulator used with the present invention possesses one or more of the following properties, among other properties:

at least one pump and at least one catheter for delivering a drug or drugs to surrounding tissue and, optionally, at least one electrode for applying stimulating current to surrounding tissue;

electronic and/or mechanical components encapsulated in a hermetic package made from biocompatible material(s);

an electrical coil or other means of receiving energy and/or information inside the package, which receives power and/or data by inductive or radio-frequency (RF) coupling to a transmitting coil placed outside the body, thus avoiding the need for electrical leads to connect devices to a central implanted or external controller;

means for receiving and/or transmitting signals via telemetry;

means for receiving and/or storing electrical power within the stimulator; and

a form factor making the stimulator implantable in a target area in the body.

A stimulator may operate independently, or in a coordinated manner with other implanted stimulators, other implanted devices, and/or with devices external to a patient's body. For instance, a stimulator may incorporate means of sensing a patient's condition, e.g., a means for sensing CHF. Sensed information may be used to control the drug and/or electrical stimulation parameters of the stimulator in a closed loop manner. The sensing and stimulating means may be incorporated into a single stimulator, or a sensing means may communicate sensed information to at least one stimulator with stimulating means.

For most patients, a continuous or intermittent stimulation throughout the day is needed to provide an adequate amount of treatment. These patients may best utilize a stimulator that has a self-contained power source sufficient to deliver repeated pulses for at least several days and that can be recharged repeatedly, if necessary. In accordance with the teachings of the present invention, the use of a stimulator with a rechargeable battery thus provides these patients the portability needed to free the patient from reliance on RF power delivery. Alternatively, the power source may be a primary battery that may last several years.

For purposes of this patent application, it is sufficient to note that RF controlled stimulators receive power and control signals from an extra corporeal antenna coil via inductive coupling of a modulated RF field. Battery-operated stimulators incorporate a power source within the device itself but rely on RF control, inductive linking, or the like to program stimulus sequences and, if a rechargeable/replenishable power source is used, to recharge/replenish the power source, when needed. In accordance with the present invention, each implanted stimulator may be commanded to produce an electrical and/or infusion pulse of a prescribed magnitude and duration and at a repetition rate sufficient to treat the targeted tissue.

For instance, stimulation may be initiated by start and stop commands from a patient-governed control switch or controller, which may be handheld, containing a microprocessor and appropriate nonvolatile memory, such as electronically erasable programmable read-only-memory (EEPROM). The controller may control the implantable stimulator by any of various means. For instance, the stimulator may sense the proximity of a permanent magnet located in the controller, or may sense RF transmissions from the controller. However, it will be evident to those of skill in circuitry and computing that many different system architectures and components could be used to achieve similar functionality with either a battery-powered or RF-powered stimulator.

### BRIEF DESCRIPTION OF THE DRAWINGS

The above and other aspects of the present invention will be more apparent from the following more particular description thereof, presented in conjunction with the following drawings wherein:

FIG. 1 is a view of the sternocostal surface of the heart;

FIG. 2 is a posterior view of the surface of the heart;

FIG. 3 illustrates an exemplary embodiment of a stimulation system of the present invention and exemplary external components of the invention; and

FIG. 4 illustrates an additional exemplary embodiment of external components of the invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

Treating congestive heart failure at as early a stage as possible offers the best chance for a longer and better quality life. Three classes of medications have been the standard treatments for heart failure: vasodilators (drugs that dilate blood vessels), particularly Angiotensin-Converting Enzyme (ACE) inhibitors; inotropics (drugs that increase the heart's ability to contract), usually digoxin; and diuretics (drugs to reduce fluid). A fourth class, beta blockers, has recently been added to the armament. The specific medication or, more commonly, a combination of these medications, is determined by the type and severity of the heart failure. Standard guidelines recommend a stepped approach to treat people with left ventricular systolic dysfunction, using an ACE inhibitor first, followed by a diuretic if the patient fails to respond to the first drug. An analysis of two trials indicated, however, that a so-called initial triple-therapy approach using an ACE inhibitor, a diuretic, and digoxin significantly prevented deterioration over four to five months compared to two-drug therapies. Side effects increase, however, and some patients may not be able to tolerate the more intensive regimen.

### Pharmacological Treatment—Acute

Vasodilators: Nitrates

Intravenous nitroglycerin (Nitro-Bid® IV, Nitrostat® IV, Tridil®) and intravenous nitroprusside (Nitropress®) are useful in short-term therapy of acute heart failure and acute pulmonary edema. Intravenous nitroglycerine tends to lose effectiveness quickly due to tachyphylaxis, but one study showed that patients who were also given oral hydralazine (another vasodilator) did not develop such a significant tolerance to this drug.

Diuretics: Loop Diuretics

Loop diuretics are used intravenously to treat pulmonary edema and acute congestive heart failure; a thiazide (another diuretic) and a loop diuretic may be administered simultaneously.

### Pharmacological Treatment—Chronic

ACE Inhibitors and Other Vasodilators

Vasodilators improve both the quality and duration of life for heart failure patients. They open the arteries and veins, thereby reducing the heart's workload and allowing more blood to reach the tissues. Vasodilators are particularly useful in treating heart failure associated with high blood pressure and dilated cardiomyopathy. Several classes of vasodilators are available, the most effective being the ACE inhibitors.

Angiotensin-Converting Enzyme (ACE) Inhibitors. These drugs block the formation of angiotensin II, a powerful enzyme that raises blood pressure, constricts blood vessels, and leads to salt retention. Although experts believe that at least 50% to 75% of patients with congestive heart failure (CHF) should be treated with ACE inhibitors, past studies have indicated that physicians are prescribing them in far fewer patients; women and nonwhites were less likely to get ACE inhibitors than white males. And when they are being prescribed, some studies indicate they are not prescribed in high enough doses to be most effective. Even worse, about 15% of patients were being prescribed expensive calcium channel blockers, which may be harmful for patients with heart failure. ACE inhibitors are of particular benefit for patients with left ventricular systolic dysfunction; those patients should take drugs indefinitely unless specific conditions make the drugs inappropriate. Some studies have indicated that ACE inhibitors improve survival over combinations of other vasodilators (hydralazine and nitrates) and might actually reduce heart damage by inhibiting the remodeling process that can cause heart attacks after heart failure. Commonly used ACE inhibitors are captopril (Capoten®), quinapril (Accupril®), enalapril (Vasotec®), lisinopril (Prinivil®), and fosinopril (Monopril®).

A persistent cough is a common and irritating side effect of ACE inhibitors. The primary adverse effect of ACE inhibitors is low blood pressure, which can be severe in some patients, particularly when therapy is initiated. Because of this, ACE inhibitors have not been used for patients who have pulmonary edema (fluid in the lungs), a condition commonly accompanied by low blood pressure. One study found, however, that these drugs may benefit even these patients, assuming that blood pressure is not excessively low to begin with. Kidney failure is a rare complication that can occur during initial therapy. Taking ACE inhibitors may also lead to excessive potassium levels, so they are not generally given with potassium-sparing diuretics or potassium supplements. (Diuretics are important for many heart failure patients, however, and diuretics that increase potassium loss are often taken with ACE inhibitors.)

Angiotensin II Receptor Antagonists. Angiotensin II receptor antagonists have benefits similar to ACE inhibitors and may have fewer or less severe side effects. ACE inhibitors are used to block the conversion of angiotensin I into angiotensin II. However, angiotensin II can still be produced by other pathways, so ACE inhibitors do not completely inhibit the deleterious effects of the renin-angiotensin system.

AT1 receptor blockers (ARBs) are used to block the renin-angiotensin system by preventing access of angiotensin II to the AT1 receptor. Because of the success of ACE inhibitors in mitigating CHF progression and because angiotensin II can still be produced by other pathways, ARBs are being vigorously evaluated for CHF. That is, since ACE inhibitors do not seem to offer complete protection against angiotensin II effects, AT1-receptor blockers may offer further advantages since the AT1 receptor mediates the known adverse effects of angiotensin II (the AT2 and other subtype receptors have no known major role). Currently, several ARBs are FDA-approved for hypertension, but none are approved for CHF.

The Evaluation of Losartan in the Elderly Study (ELITE-1) compared the ARB losartan to the ACE inhibitor captopril in patients aged over 65 years with at least NYHA class II and left ventricular ejection fraction (LVEF) greater than 40%. The ARB group had trends toward lower mortality (4.8% vs. 8.7%, p=0.035), less hospitalization, less discontinuance due to cough, and equivalent effect on renal dysfunction. This study and others have been used as rationale for ARB use in CHF. However, the larger ELITE-II study confirmed statistically equivalent, but not superior, mortality rates. The clinician is left with no clearly supportive data for the superiority of ARB use in CHF, except in patients who are intolerant to ACE inhibitors. A number of ongoing mortality studies are using several ARBs in NYHA class II-IV CHF, both compared with and in combination with ACE inhibitors. Since they operate by different mechanisms, ARBs and ACE inhibitors may be complementary in CHF treatment. However, this is speculative, so results of well-designed clinical trials are eagerly awaited.

Hydralazine and Nitrates. The oral direct-acting vasodilators hydralazine and isosorbide dinitrate improve symptoms and may prolong life when used in combination. Isosorbide mononitrate, an organic nitrate and the major biologically active metabolite of isosorbide dinitrate, is a vasodilator with effects on both arteries and veins. Nitrates are used to treat the chest pain associated with angina and to ease the symptoms of congestive heart failure (CHF). Intravenous nitroglycerin (Nitro-Bid® IV, Nitrostat® IV, Tridil®) and intravenous nitroprusside (Nitropress®) are useful in short-term therapy of acute heart failure and acute pulmonary edema. Intravenous nitroglycerine tends to lose effectiveness quickly due to tachyphylaxis, but one study showed that patients who were also given oral hydralazine did not develop significant tolerance to this drug. Combinations of hydralazine with nitrates are more effective than either drug used alone and are recommended when patients cannot tolerate ACE inhibitors.

Amyl Nitrite. Amyl nitrite is a rapidly acting vasodilator. Amyl nitrite causes a non specific relaxation of smooth muscle with the most prominent actions occurring in vascular smooth muscle. This effect on vascular smooth muscle results in coronary vasodilation and decreased systemic vascular resistance and left ventricular preload and afterload. Myocardial ischemia is relieved in patients with angina pectoris, with an abatement of chest pain and possibly other related symptoms. It can be used to treat CHF by acutely reducing afterload.

Calcium-Channel Blockers. Calcium channel blockers are vasodilators commonly used for high blood pressure and angina. Short-acting calcium channel blockers, however, may worsen heart failure. Recent studies have also found a number of other adverse effects. Unfortunately, they are currently over-prescribed for patients with heart failure. A newer generation calcium-channel blocker, amlodipine (Norvasc®), may reduce death rates in a small subgroup of heart failure patients who have idiopathic dilated cardiomyopathy without coronary artery disease.

Inotropic Drugs and Digitalis

Until recently, the inotropic drug digitalis was the first-line therapy for heart failure. Digitalis increases the strength of the heart's contractions, reduces heart size, and reduces certain arrhythmias. Derived from the foxglove plant, it has been used to treat heart disease since the 1700s and is still the only oral inotropic agent in general use. Controversy has been ongoing for more than 100 years over whether the benefits of digitalis outweigh its risks and adverse effects. In general, digitalis does not reduce mortality rates, although it improves symptoms. Patients who take digitalis are also hospitalized slightly less often than those not taking the drug. Many experts now believe that patients should first be prescribed drugs proven to prolong life, such as an ACE inhibitor or a new generation beta blocker, such as carvedilol, before digitalis is recommended. Digitalis may be useful for patients with systolic dysfunction characterized by low ejection fractions and is helpful in heart failure patients with atrial fibrillation—a type of rapid, irregular heartbeat. Digitalis may even be harmful in some patients with heart failure, particularly when caused by diastolic dysfunction characterized by normal to high ejection fraction.

Digoxin (Lanoxin®). Digoxin is the most commonly prescribed digitalis preparation. While digitalis is generally a safe drug, it can have toxic side effects caused by overdose or other accompanying conditions. The most serious side effects are arrhythmias—abnormal heart rhythms that can be life-threatening. Factors which increase the risk of toxicity include advanced age, low blood potassium levels (which can be caused by diuretics), hypothyroidism, anemia, valvular heart disease, and impaired kidney function. Digitalis interacts with many other drugs, including, but not limited to, quinidine, amiodarone, verapamil, flecainide, amiloride, and propafenone. Early signs of toxicity may be irregular heartbeat, nausea, vomiting, stomach pain, fatigue, visual disturbances, and emotional and mental disturbances. Some of these side effects may be mild and not harmful. Toxic side effects used to be experienced by nearly 25% of patients taking digitalis, but now that a blood test can be used to monitor the level of the drug in the blood, toxicity is down to 2%. A recent study reported that for most patients with mild to moderate heart failure, low-dose digoxin may be as effective as higher doses. It was found that patients who stopped taking digoxin after using it in combination with ACE inhibitors were at risk for worsening heart failure.

Other Inotropic Drugs. There was a surge of interest in other oral inotropic agents, including vesnarinone, milrinone, flosequinan, and inamrinone. Large studies of these agents, however, were disappointing and some even reported increased mortality rates.

Diuretics

Diuretics have long been used to relieve fluid retention, a hallmark of CHF. Aggressive use of diuretics, even in people taking ACE inhibitors, can reduce hospitalizations and improve exercise capacity. Diuretics act on the kidneys to rid the body of excess salt and water. They reduce the accumulation of fluid in the legs, abdomen, and lungs, lower blood pressure, and improve the efficiency of the circulation. Side effects of diuretics include low blood pressure, dehydration, and kidney dysfunction; they also may trigger gout, increase blood sugar and triglyceride, LDL, and overall cholesterol levels, and may deplete the B vitamin thiamin. Although many diuretics are available, they are generally categorized as thiazides and loop diuretics, used with or without potassium-sparing agents. It is important to note that a recent study found an increased incidence of hospitalization in patients who were taking nonsteroidal anti-inflammatory drugs (NSAIDs) along with diuretics. Common NSAIDs include aspirin, ibuprofen, and naproxen.

Thiazides. Thiazides, including hydrochlorothiazide (HydroDIURIL®, Esidrix®), chlorothiazide (Diuril®), metolazone (Zaroxolyn®), and chlorthalidone (Hygroton®), are usually prescribed for patients with mild heart failure and good kidney functioning.

Loop Diuretics. Loop diuretics, such as furosemide (Lasix®), bumetanide (Bumex®), and ethacrynic acid (Edecrin®), are generally used for more severe heart failure, especially when kidney function is impaired. Loop diuretics are used intravenously to treat pulmonary edema and acute CHF; a thiazide and a loop diuretic may be administered simultaneously. Fluid may persist in the lungs even after standard treatment for congestive failure, limiting the patient's ability to function normally. One study treated patients with this condition very aggressively with furosemide to further reduce fluids, but no improvement was seen. Another method using a filtration technique was more successful.

Potassium-Sparing Agents. Potassium loss is a major problem with diuretic use. Unless patients are also taking ACE inhibitors, which raise potassium levels, the physician may recommend a potassium supplement or the use of a potassium-sparing diuretic, such as spironolactone (Aldactone®), amiloride (Midamor®), and triamterene (Dyrenium®), along with a thiazide or loop diuretic. All patients receiving diuretics with or without potassium-sparing drugs should have their blood potassium levels checked at regular intervals.

Beta Blockers

Beta blockers prevent norepinephrine (adrenaline) from binding to heart cells, which affects the frequency and force of heartbeats. Elevated levels of norepinephrine are associated with severe heart failure. Because beta blockers also reduce the pumping action of the heart in the short term, they have not been used until recently for treatment of heart failure.

Carvedilol and Other Non-Selective Beta Blockers. Carvedilol (Coreg®) is known as a nonselective beta blocker and is proving to have important benefits for many patients, including patients with mild to even very severe heart failure. It appears to have vasodilating and antioxidant properties. If administered after a first heart attack, it may even help prevent left ventricular remodeling—one of the damaging processes leading to heart failure. Combinations of this beta blocker with other heart failure medications can improve heart function and size and even reduce mortality rates in some patients. Its positive effect on symptoms, including the ability to perform physical exercise, however, is not as apparent. Carvedilol must be monitored and the dosages regulated very carefully, however, since heart failure may actually worsen in the early stages of treatment. Those at higher risk for worsening heart failure appear to be those with lower systolic blood pressure and lower sodium levels in the blood. Most of these events occur within six weeks of starting the drug with more than half occurring within two weeks when patients are on the lowest dose. It should not be used in people with asthma, those with very slow heartbeats (bradycardia), patients on intravenous inotropics, or people with certain heart conduction disorders. Although it appears to be effective for heart failure from nearly any cause, the drug may be more useful for certain people, including those with dilated cardiomyopathy, and patients with fast heart rates (faster than 82 beats per minute). Bucindolol is a similar beta blocker under investigation.

Selective Beta Blockers. Studies are finding that some older and less expensive beta blockers called selective beta blockers may also significantly reduce mortality rate in heart failure patients. Such agents include metoprolol (Lopressor®) and bisoprolol (Zebeta®). One study comparing metoprolol with carvedilol reported significant improvement in both groups and no difference in the effects of the two drugs. In both patient groups, walking distance and ejection fractions improved to the same degree. This was a small study conducted for only six months. Another study reported that bisoprolol improved survival rate in patients with moderate heart failure. Experts also warn that because of the increased dangers during early treatment, the widespread use of beta blockers for heart failure warrants caution and these drugs, including carvedilol, should be administered only by specialists experienced in treating heart failure.

Anti-Clotting Drugs

Two studies have reported that heart failure patients taking drugs that prevent blood clots, including warfarin (Coumadin®), aspirin, or dipyridamole (Persantine®), have a significantly reduced risk of death. Both studies were primarily investigating the ACE inhibitor enalapril, and in one of the studies, patients who took enalapril along with aspirin or dipyridamole did slightly less well than those taking the anti-clotting drugs alone (but still did better than patients who were taking none of these drugs). Experts warn, however, that until more studies are done, anti-clotting drugs should be used with caution in heart failure patients unless they have atrial fibrillation, previous thromboembolism, or other risk factors for blood clots.

Drugs for Arrhythmias

Drugs used to treat irregular heartbeats (arrhythmias), which are a particular danger for congestive heart patients, have not been very successful in prolonging survival when used as part of the treatment regimen for CHF. Trials of the anti-arrhythmic drug amiodarone (Cordarone®) have reported improved mortality rates in patients with severe heart failure who also had very rapid heart rates and also in those with atrial fibrillation, a condition marked by rapid twitching of the heart walls. In some studies, not only was there a reduction in death rate, but the patients' ability to function was also improved. The drug apparently has no benefit for those with slower heart rates.

Other Drugs for Relief of Symptoms

Ipratropium (Atrovent®), a drug normally used by asthma patients, was tested in a small study of smokers and nonsmokers with CHF for improving lung function. Breathing improved in all patients who were administered four puffs of the drug using an inhaler. The drug has no known adverse effects on the heart, and there were no other side effects in this group. More studies are needed. Another asthma drug, theophylline, was found to improve oxygen levels and lung function in heart failure patients who also experienced central sleep apnea, the disordered breathing syndrome associated with left-side heart failure.

Additional Related Medications

Antithrombotic Agents. Ischemic heart disease is a common cause of CHF, and ischemic heart disease is typically treated with antithrombotic agents, in addition to other possible medications. Medications such as aspirin, warfarin, and heparin are used to treat ischemic heart disease, and by decreasing ischemic cardiac events, antithrombotic medications are likely to slow the progression of CHF.

Antiarrhythmic Agents. Patients with CHF may also suffer from a cardiac arrhythmia due to the same underlying cardiac pathology (e.g., ischemic heart disease). An arrhythmia may reduce cardiac output and exacerbate symptoms of CHF. In CHF patients with a cardiac arrhythmia, an antiarrhythmic agent such as sotalol, dofetilide, or amiodarone may be used to treat the comorbid arrhythmia condition.

Antihyperlipidemic Agents. An increased blood level of low density lipoprotein (LDL) cholesterol and a decreased blood level of high density lipoprotein (HDL) cholesterol heart disease is present in a number of patient with CHF. A decrease in LDL cholesterol level and an increase in HDL cholesterol level is believed to slow the progression of ischemic heart disease and may similarly slow the progression of CHF. Antihyperlipidemic agents such as lovastatin, pravastatin, simvastatin, fluvastatin, atorvastatin, cerivastatin, niacin, cholestyramine, and colestipol are used to lower the LDL cholesterol level and increase the HDL cholesterol level in CHF patients who have abnormal cholesterol levels. (Lovastatin, pravastatin, simvastatin, fluvastatin, atorvastatin, and cerivastatin are collectively referred to as statin drugs, since their mechanism of action is the same.) Drugs such as niacin, clofibrate, gemfibrozil, and fenofibrate also may reduce blood levels of triglycerides, which may also slow the progression of ischemic heart disease and CHF.

### Experimental and Emerging Medications

Vasopressin Antagonists

Circulating arginine vasopressin (AVP) is a potent vasoconstrictor, and elevated levels of AVP may lead to systemic vasoconstriction and contribute to the hyponatremia often seen in patients with advanced CHF. AVP levels are increased to approximately twice the normal level in many patients with symptomatic heart failure. Loop and proximal tubule diuretics (e.g., Lasix®) are common treatments to ameliorate, among other effects, elevated AVP, but their use is limited by electrolyte loss, reduction of glomerular filtration rate, and further activation of the renin-angiotensin-aldosterone system. Vasopressin antagonists, or vaptans, are a novel class that are devoid of many of the negative effects of loop diuretics. This has excited interest in AVP antagonism as CHF therapy.

AVP activity is mediated through 2 receptors, V1 (consisting of V1A and V1B) and V2. The V1A receptor is found on blood vessels and in the myocardium, where it functions as a strong vasoconstrictor and myocardial stimulant, respectively. The V1B receptor only has indirect circulatory effects by way of brainstem-mediated baroreceptor function and does not appear to be as major a cardiovascular target. The V2 receptor mediates the effects of AVP on water excretion by coupling with the aquaporine channels in the renal collecting tubules. AVP antagonists could be of clinical benefit by reducing systemic vascular resistance and increasing cardiac output in patients with heart failure and elevated AVP. Nonselective V1A/V2 nonpeptide antagonists have been shown to inhibit the pressor response to AVP (V1A activity) and increase urine volume, with reduced urine osmolality (V2 activity). Selective V2 receptor antagonists are also being developed, and it has not been determined which approach will be most effective. In animal models of CHF, the vaptans induce aquaresis and decrease pulmonary congestion and LV end-diastolic pressure. Importantly, aquaretic effects are maintained in CHF with minimal renal electrolyte loss, and there is no further activation of the sympathetic nervous or renin-angiotensin-aldosterone systems.

Human phase 1 clinical trials have been generally without serious adverse events or electrolyte or electrocardiographic changes. The most common side effects were thirst and diuresis. Phase 2 and 3 clinical trials are ongoing with several proprietary vasopressin antagonists being studied in NYHA class II-IV CHF patients already receiving contemporary CHF therapy. Both oral and IV forms are being examined as adjuncts to diuretics both during acute exacerbations and for the long term.

Tumor Necrosis Factor (TNF) Immunotherapy

Although neurohormonal activation has been the focus of therapy in CHF for years, there is another novel group of biologically active molecules, the pro-inflammatory cytokines that are activated in CHF. Pro-inflammatory cytokines are a class of secretory polypeptides synthesized and released by macrophages, leukocytes, and endothelial cells in response to injury. Patients with CHF overexpress specific pro-inflammatory cytokines, including tumor necrosis factor (TNF) and interleukins 1 and 6 (but these are less well studied). Cytokines exacerbate hemodynamic abnormalities and/or exert toxic effects by binding specific cell surface receptors.

Experimentally, inducing high levels of TNF can produce many of the characteristics of heart failure, such as progressive LV dysfunction, remodeling, and cardiomyopathy. TNF is not constitutively expressed in a hemodynamically unstressed adult heart, but is rapidly synthesized by the heart in response to hypertrophic stress stimuli. TNF levels return to normal after the hemodynamic stress is removed. Patients with advanced heart failure have increased TNF levels, and a direct correlation exists between TNF levels and deteriorating NYHA functional class. Many of the features of CHF are compatible with (or at least can be exacerbated by) the biologic effects of TNF, which induces the release of other pro-inflammatory cytokines and can act as an endocrine hormone contributing to cachexia. The mechanism by which TNF exerts its abnormal effects may be by preventing a rise in intracellular calcium or by stimulating nitric oxide production. Therefore, therapies designed to block TNF production or receptor binding may be helpful in mitigating TNF activity in the failing heart.

Etanercept is an antibody-based immunotherapy made up of 2 TNF receptors fused to the Fc portion of IgG1. As such, it binds TNF and makes it biologically inactive. A major contraindication to its use is the presence of any nidus of infection that may lead to sepsis. In fact, as more information is becoming available, close surveillance for any sign of infection is becoming more important for actively treated patients. Also, administration is subcutaneous, which may be difficult for some patients. Etanercept is already FDA-approved for treating advanced rheumatoid arthritis, but not any cardiac condition. Two phase-1, placebo-controlled, clinical safety trials have shown that this anti-TNF antibody is generally well tolerated in patients with NYHA class III-IV (LVEF<35%) while lowering serum TNF levels. One of these pilot trials suggested improvements in LV function and improvement in symptoms. Of course, the total burden of cytokines in the pathophysiology of CHF remains to be determined. Currently, large, randomized clinical trials are examining anti-TNF immunotherapy in NYHA class II-III CHF to examine effects on mortality, clinical status, and quality of life.

Neutral Endopeptidases and Vasopeptidases

Atrial natriuretic peptide (ANP) and brain natriuretic peptide (BNP)—a misnomer since BNP is predominantly of ventricular origin in CHF—are cardiac hormones that are synthesized, stored, and released initially from atrial and ventricular cardiocytes in response to abnormal cardiac pressure and volume overload. Circulating biologically active ANP and BNP bind to the natriuretic peptide-A receptor located on endothelial and vascular smooth muscle cells, which in turn mediate natriuresis, vasodilation, and renin inhibition. Circulating ANP and BNP levels are elevated in CHF, and these levels are directly correlated with the functional class of heart failure. These cardiac hormones function to counter many of the vasoconstriction and salt- and water-retaining effects of the adrenergic, renin-angiotensin, and AVP systems. That is, ANP and BNP attempt to protect the central circulation from volume overload. ANP and BNP are rapidly degraded by the ectoenzyme neutral endopeptidase (NEP) and are cleared by the unique cell-surface natriuretic peptide-C receptor. Therapeutic strategies for heart failure being clinically developed include infusing ANP and/or BNP or, more practically, inhibiting NEP to increase circulating ANP and BNP.

One class of agents, vasopeptidase inhibitors, combines inhibition of NEP with inhibition of ACE. One such drug, omapatrilat, has antihypertensive action and is undergoing investigation for heart failure. The question is whether such agents offer advantages in terms of LV remodeling over ACE inhibition alone. Other strategies include pure NEP enzyme inhibition; ecadotril and candoxatril are two such investigational drugs.

Endothelin Receptor Antagonists

Endothelin is a powerful vasoconstrictor that possesses antinatriuretic and mitogenic actions that may worsen CHF. The endothelin family includes a group of three 21-amino acid peptides with very similar structures: endothelin-1 (ET-1), ET-2 and ET-3. Increasing evidence suggests a potential role of the endothelin system in the pathophysiology of CHF.

ET-1, the predominant endothelin isoform, is the most potent vasoconstrictor identified to date. ET-1 is produced throughout the cardiovascular system and, acting as both an autocrine and paracrine hormone, triggers intracellular events that promote pathologic growth and hypertrophy in the myocardium. ET-1 is usually cleared rapidly by the renal, hepatic, and pulmonary systems. ET-1 levels are two to three times higher in patients with heart failure and have prognostic significance since they are inversely correlated with LVEF and cardiac index, and are directly correlated with NYHA functional class. Indeed, increased ET-1 after myocardial infarction predicts reduced one-year survival.

With the recent discovery and development of endothelin receptor antagonists, the clinical potential of therapeutic agents that target the endothelin system is being actively evaluated. Extensive preclinical trials show that antiendothelin therapy improves hemodynamics (both acutely and chronically), left ventricular remodeling, neurohumoral activation, and renal dysfunction, and decreases mortality risk.

The effects of endothelin are mediated by two different receptors: ET_{A}, which mediates vasoconstriction, and ET_{B}, which causes both constriction and vasodilation. The ETA receptor is a G-protein coupled receptor found primarily on vascular smooth muscle cells but also on cardiocytes and fibroblasts. ETB receptors are found on the vascular endothelium and mediate nitric oxide-dependent vasodilation, ET-1 synthesis, and clearance of ET-1 from the circulation. Some endothelin receptor antagonists block the ET_{A} receptor and others block both ET_{A} and ET_{B} receptors. Endothelin receptor antagonists being examined for treatment of CHF include darusentan, sitaxsentan, tezosentan, and bosentan. Sitaxsentan and tezosentan are available only for intravenous use.

Bosentan, an orally active endothelin receptor antagonist, is to date the most well studied of these agents for the treatment of CHF. Bosentan improves hemodynamics, left ventricular function, and cardiac remodeling in animal models of chronic heart failure. Several factors may account for the cardioprotective effects of bosentan, including reduced cardiac preload and afterload, improved coronary blood flow, inhibition of neurohormonal activation, and chronic structural effects (inhibition of cardiac remodeling, cardiac hypertrophy and cardiac fibrosis) by direct inhibition of the actions of ET-1 on myocardial cells. In animal experiments, treatment with bosentan has been associated with beneficial pharmacological effects, including vasodilation, prevention of cardiac remodeling, and improvement of ventricular performance.

Selective Aldosterone Receptor Antagonists

The renin-angiotensin-aldosterone system is chronically activated in CHF. Even high doses of ACE inhibitors may not completely inhibit the complete axis, since aldosterone acts through mineralocorticoid receptors. The Randomized Aldactone Evaluation Study (RALES) examined the addition of spironolactone, a competitive aldosterone receptor antagonist, to ACE inhibition plus standard therapy in patients with NYHA class III-IV CHF. This trial was stopped prematurely for efficacy with a 30% decrease in mortality and hospitalization. However, spironolactone has frequent steroid-related endocrine side effects including gynecomastia, loss of libido, and menstrual irregularities. As such, selective aldosterone receptor antagonists are being developed that have decreased affinities for androgen and progesterone receptors. One compound, eplerenone, is currently undergoing clinical trials.

### Pacemaker Therapy

Although drug therapy is the primary therapy for patients with heart failure, the use of pacemakers to improve cardiac hemodynamics is under investigation. Despite promising initial results, controlled studies have not verified the benefit of VDD (ventricular pacing with dual-chamber sensing) or DDD (dual-chamber pacing with dual-chamber tracking) pacing to a nonselected population of severely symptomatic CHF patients to shorten their atrioventricular (AV) delay. There is increasing interest in pacing the left side of the heart or simultaneously pacing the right and left ventricles. Early studies suggest that these techniques may produce favorable hemodynamic effects in patients with CHF. Controlled, randomized studies are now underway. Further, it has been shown that sudden cardiac death accounts for 50% of deaths in patients with CHF. The value of an implantable cardioverter defibrillator (ICD) in secondary prevention of sudden cardiac death is well established. The use of ICDs for primary prevention of sudden cardiac death in patients with CHF is being actively evaluated. Several large multicenter trials are underway, some combined with biventricular pacing, and should provide useful data in the coming years.

### Surgical Treatment

While drug therapy is the most common approach to heart failure, certain patients may require surgical interventions. Patients with severe coronary artery disease may benefit from angioplasty or bypass surgery. Patients with faulty heart valves can have artificial valve replacement surgery.

Heart Transplantation

Class III and IV patients who suffer from severe heart failure and whose symptoms do not improve with drug therapy may be candidates for heart transplantation. Traditionally transplants have been performed only on patients under 60 years of age, but studies indicate that selected older patients can benefit from this procedure and one study found that older transplant patients achieved a better quality of life than younger patients. While the risks of this procedure are high, the two-year survival rate is about 78% and after five years it ranges from 50% to over 70%. About 76% of transplant patients are male and 85.4% are white. In general, the highest risk factors for death three or more years after a transplant operation are coronary artery disease and the adverse effects (infection and certain cancers) of immunosuppressive drugs used in the procedure. Older patients are at particular risk for cancers from these drugs and for osteoporosis, but their rejection rates appear to be similar to those of younger patients.

Alternatives to or Holding Measures Until Transplantation

In any one month, about 4,000 people are registered for a heart transplant procedure, although only about 166 transplants are performed each month. A number of procedures are now available for patients who await transplantation; some may even offer permanent alternatives. Studies indicate that most patients in stable condition can be managed safely with medications for many months while waiting for a transplant. Portable pumps that continuously infuse medications such as dopamine and prostaglandin E-1 can allow the patient to remain mobile and active. Other procedures may also be performed to help maintain heart function.

Implanted Devices. In those whose heartbeat has slowed dangerously (called bradycardia), a left ventricular assist device (LVAD) may be implanted in the chest to increase the pumping action of the heart. Until recently, LVAD required a large, hospital-based immobile console to which the patient was attached while waiting for a transplant. Miniaturized battery-powered LVAD units, however, may allow many patients to leave the hospital and even resume normal activities, such as work, golf, and sexual activity. Eventually these smaller devices may even provide a permanent solution for some patients who are not candidates for transplantation. Some evidence exists that they may even restore function in failing heart cells. There are risks, however, involved with LVADs, including bleeding, blood clots, and right side heart failure. Devices called implantable cardioverter-defibrillators (ICDs) may be effective for preventing arrhythmias in heart failure patients.

Ventricular Remodeling. Ventricular remodeling (also called partial left ventriculectomy or the Batista procedure, after its inventor) may allow some patients with dilated cardiomyopathy to avoid a heart transplant. The procedure involves first removing a section of healthy heart muscle (weighing about three ounces). The surgeon then reshapes the heart to a more normal size and form and repairs any faulty heart valves. The procedure is effective in about 75% of cases; unfortunately, if it fails, the patient must have an immediate heart transplant. The procedure is not used for people whose heart failure developed from coronary artery disease or after a heart attack. Ventricular remodeling is a new procedure and not yet widely available; early trials in the U.S. have reported positive results and improvement has been sustained in the Brazilian patients whose hypertrophy resulted from valvular or viral disease. In cases where the muscle itself was diseased, the hearts have begun to redilate. Even in such cases, however, the procedure can be repeated.

Dynamic Cardiomyoplasty. Dynamic cardiomyoplasty is useful in carefully selected patients with CHF. In this procedure, one end of a muscle from the patient's back is detached and wrapped around the ventricles of the heart. After a few weeks, these relocated muscles are conditioned with electrical stimulation to behave and beat as if they were heart muscles. The procedure benefits the failing heart in many ways, including improving systolic pressure, limiting dilation of the heart, and reducing heart muscle stress. Oddly, the procedure seems to improve symptoms significantly although it has only a modest effect on actual blood circulation. One long-term study reported that heart muscle structure was preserved in 84% of patients. Survival rates were estimated to average 54% after seven years, but they may be higher or lower depending on individual characteristics. The procedure does not preclude a later transplant operation.

Intra-aortic Balloon Pump. The intra-aortic balloon pump (IABP) is a device that is helpful for maintaining heart function for people awaiting transplants. It is usually used for short periods, but recent studies indicate that patients may be able to use it safely for somewhat longer periods (an average duration of 23 days in one study).

### Gene Therapy

Due to improvements in vector technology, cardiac gene delivery, and understanding of the molecular pathogenesis of heart failure, gene therapy for heart failure merits consideration at this time. Gene transfer may also provide an important tool for validating specific targets. Several interventions, particularly those enhancing sarcoplasmic calcium transport, show promise in animal models of heart failure and in myopathic cardiomyocytes derived from patients. [See Hajjar, et al. “Prospects for gene therapy for heart failure” Circulation Research 2000 Mar. 31; 86(6):616-21.]

In human and experimental models of heart failure, sarcoplasmic reticulum Ca(2+) ATPase (SERCA2a) activity is decreased, resulting in abnormal calcium handling. The disturbances in calcium metabolism have been shown to contribute significantly to the contractile dysfunction observed in heart failure. An investigation was conducted into whether increasing SERCA2a expression can improve ventricular function in an animal model of heart failure. At least one investigation has shown that, in an animal model of heart failure where SERCA2a protein levels and activity were decreased and severe contractile dysfunction was present, overexpression of SERCA2a in vivo restored both systolic and diastolic function to normal levels. [See Miyamoto, et al. “Adenoviral gene transfer of SERCA2a improves left-ventricular function in aortic-banded rats in transition to heart failure.” Proceedings of the National Academy of Sciences of the United States of America 2000 Jan. 18;97(2):793-8.]

A pseudophosphorylated mutant of human phospholamban, delivered by an in vivo recombinant adeno associated virus (rAAV) vector, penetrated 60% of the myocytes in hamsters with heart disease. Phospholamban is a regulatory protein that controls calcium entry into the heart muscle. The treatment enhanced myocardial SERCA2a uptake and suppressed progressive impairment of left ventricular (LV) systolic function and contractility for 28-30 weeks, thereby protecting cardiac myocytes from cytopathic plasma-membrane disruption. [See Hoshijima “Chronic suppression of heart-failure progression by a pseudophosphorylated mutant of phospholamban via in vivo cardiac rAAV gene delivery” Nature Medicine 2002 Aug;8(8):874-71.]

### Sensing Cardiac Function

A number of means are available for assessing cardiac function. An ultrasound echocardiogram can non-invasively assess a number of parameters of the heart, such as left ventricle size and cardiac output. An electrocardiogram (ECG) may be recorded non-invasively or invasively, and may be used to detect or diagnose a number of cardiac conditions, e.g., ischemia, arrhythmia, etc. Invasive pressure transducers may be used to determine left ventricular end diastolic pressure, pulmonary capillary wedge pressure, and systemic blood pressure. For instance, a thermal dilution catheter, the dye-dilution method, and/or catheter pressure transducers/catheter tip transducers may be used to measure blood pressure or cardiac output. Cardiac output, the total volume of blood pumped by the ventricle per minute, is the product of heart rate and stoke volume.

In a 1990 study of 21 heart transplant patients, Pepke-Zaba, et al. compared cardiac output measured by thermodilution and by impedance cardiography. They found close agreement between the measurements, both at rest and during exercise. Both measurements followed changes in heart rate and oxygen consumption. Both thermodilution and impedance cardiography methods elicited good reproducibility of cardiac output measurements at rest and during exercise. The authors concluded that the noninvasive and continuous record of cardiac output obtained by impedance cardiography can be used for the monitoring of cardiac output. [See Pepke-Zaba, et al. “Validation of impedance cardiography measurements of cardiac output during limited exercise in heart transplant recipients.” Transplant International 1990 July;3(2):108-12.]

According to the present invention, congestive heart failure is treated via an implantable pump and catheter(s) used to deliver one or more stimulating drugs, plus, optionally, an implantable signal generator and electrode(s) to deliver electrical stimulation to the target area(s). One or more catheters are surgically implanted in one or more of the coronary arteries, coronary veins, aorta, ventricles, atria, pulmonary arteries, and pulmonary veins to infuse the stimulating drug(s), and, optionally, electrode(s) on a lead(s) are implanted to provide electrical stimulation.

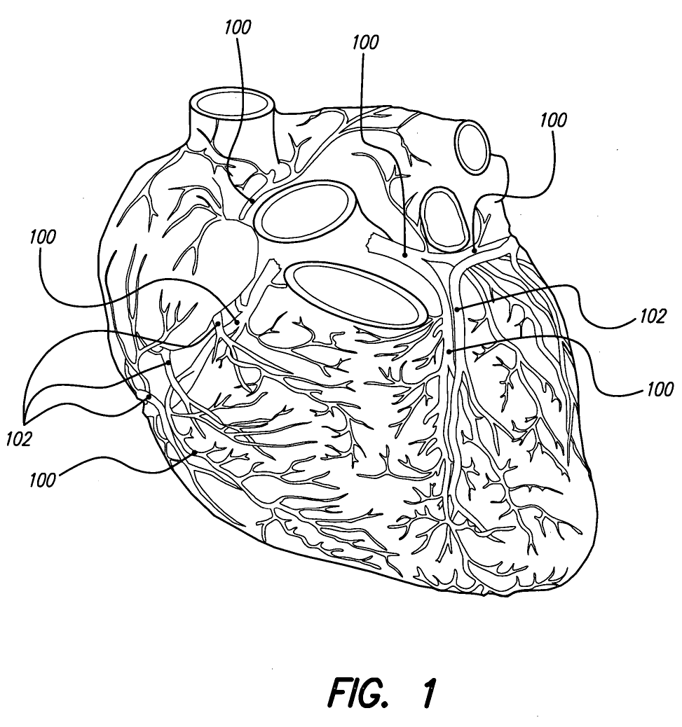

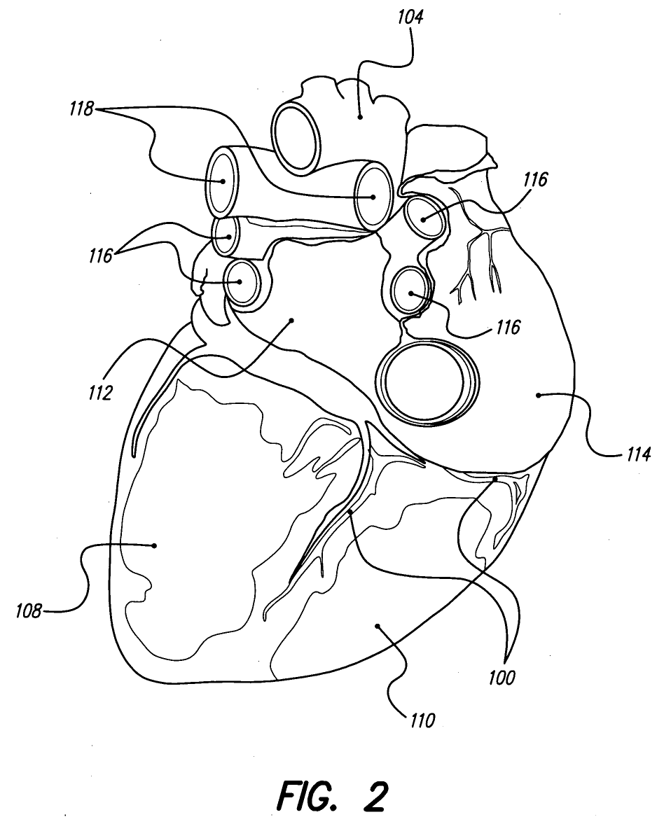

FIG. 1 depicts the coronary arteries and cardiac veins of the sternocostal surface of the heart, while FIG. 2 is a posterior view of the diaphragmatic surface of the heart. Drugs to treat CHF may be delivered to one or more of the coronary arteries 100 (which herein describes also branches of the coronary arteries), one or more of the coronary veins 102 (also including branches), the aorta 104, the left ventricle 108, the right ventricle 110, the left atrium 112, the right atrium 114, one or more of the pulmonary veins 116, and one or more of the pulmonary arteries 118. Electrical stimulation may also be applied during infusion of one or more stimulating drugs.

As indicated above, the present invention is directed to treating CHF and relieving its symptoms. In accordance with the teachings of the present invention, one or more stimulating drugs, possibly combined with electrical stimulation, are applied to one or more of the above mentioned areas for such treatment. As used herein, stimulate, stimulation, and stimulating refer to infusion of a stimulating drug(s) and/or supplying electrical current pulses. As such, infusion parameters and/or electrical current parameters are sometimes referred to herein as simply stimulation parameters, which parameters may include amplitude, volume, pulse width, infusion rate, and the like. Similarly, stimulation pulses may be pulses of electrical energy and/or pulses of drugs infused by various means and rates of infusion, such as intermittent infusion, infusion at a constant rate, and bolus infusion.

As used herein, stimulating drugs comprise medications and other pharmaceutical compounds, anesthetic agents, synthetic or natural hormones, neurotransmitters, interleukins, cytokines, lymphokines, chemokines, growth factors, and other intracellular and intercellular chemical signals and messengers, and the like. In addition, stimulation of an area herein may include stimulation of cell bodies and axons in the area.

The invention includes at least one stimulator. In the case of drug infusion only, a stimulator may comprise an implantable pump. In the case of electrical stimulation, as well, the stimulator may also comprise an implantable pulse/signal generator (IPG). In cases where both electrical stimulation and drug infusion are required or desired, more than one stimulator may be used. Alternatively, a stimulator may provide both electrical stimulation and one or more stimulating drugs.

The present invention includes a stimulator that may be implanted in a surgically-created shallow depression or opening in the thorax, abdomen, or above the buttock, that may conform to the profile of surrounding tissue(s) and/or bone(s), and is small and compact. This may minimize any cosmetic impact, and minimize pressure applied to the skin or other tissue, which pressure may result in skin erosion or infection. As such, a stimulator of certain embodiments of the present invention has a diameter of about 75 mm, or only about 65 mm, or even less than about 55 mm. In such configurations, stimulator thickness may be about 10-12 mm, or even less than 10 mm.

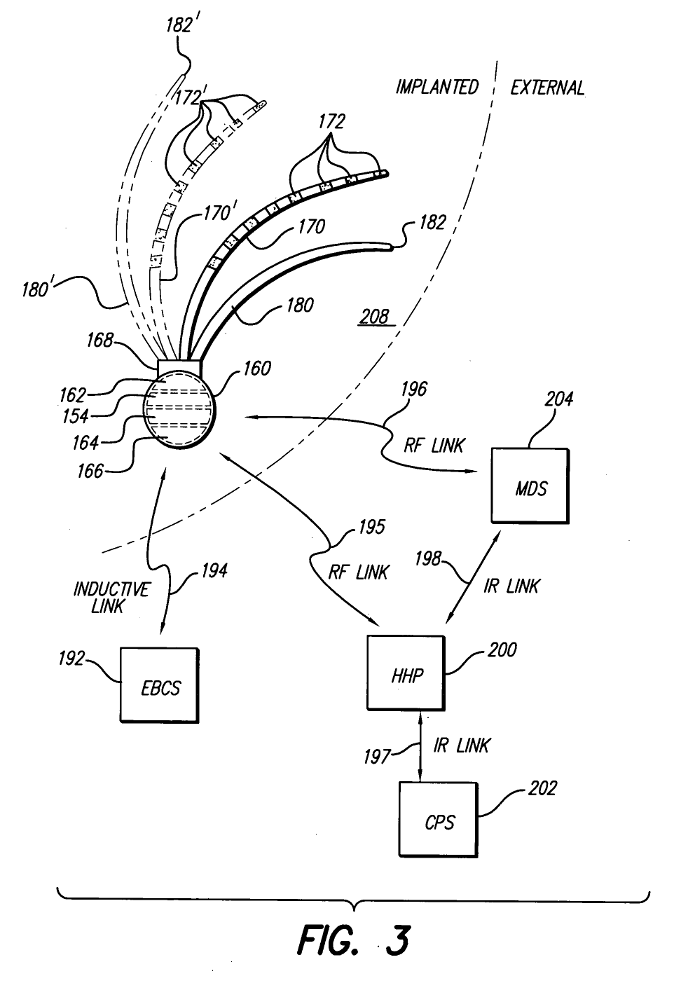

In the exemplary embodiment of FIG. 3, one or more catheters 180 and, optionally, one or more leads 170 attach to stimulator 160 and run subcutaneously, such as in a surgically-created tunnel(s), to the tissues to be stimulated. In the case of treatment including electrical stimulation, electrode(s) 172 are carried on lead 170 having a proximal end coupled to stimulator 160. Electrode(s) 172 may include, for instance, a tip electrode and/or one or more ring electrodes, allowing, e.g., temporally synchronized stimulation. The lead contains wires electrically connecting electrodes 172 to stimulator 160. Stimulator 160 contains electrical components 154 that produce electrical stimulation pulses that travel through the wires of lead 170 and are delivered to electrodes 172, and thus to the tissue surrounding electrodes 172. Implantation of such stimulators, leads, and catheters in the locations specified herein is performed as known to those in the art, e.g., as known to interventional cardiologists.

In the case of treatment alternatively or additionally constituting drug infusion, catheter(s) 180 are coupled at a proximal end to stimulator 160, which contains at least one pump 162 for storing and dispensing one or more drug(s) through the catheter(s) 180. At or along a distal end, catheter 180 has at least one discharge portion 182 for infusing dosages of the one or more drugs into a predetermined site. Catheter 180 may also act as a lead, additionally including electrode(s) 172 at and/or along its distal end.

To protect the components inside stimulator 160, some or all of the case of the stimulator may be hermetically sealed. For additional protection against, e.g., impact, the case may be made of metal (e.g. titanium), ceramic, or the like, which materials are also, advantageously, biocompatible. The material comprising the case of the stimulator 160 may be chosen to limit passage of water vapor, while permitting passage of electromagnetic fields used to transmit data and/or power. In addition, stimulator 160 may be configured to be Magnetic Resonance Imaging (MRI) compatible.

According to embodiments as depicted in FIG. 3, at least one lead 170 and/or catheter 180 is attached to stimulator 160, via a suitable connector(s) 168, if necessary. Each lead includes at least one electrode 172, and may include as many as sixteen or more electrodes 172. Additional leads 170′ and/or catheter(s) 180′ may be attached to stimulator 160. Hence, FIG. 3 shows (in phantom lines) a second catheter 180′, having discharge portion 182′, and a second lead 170′, having electrodes 172′ thereon, also attached to stimulator 160.

Lead(s) 170/170′ may, for instance, be less than about 5 mm in diameter, or may be even less than about 1.5 mm in diameter. Electrodes 172, 172′ may be made of a noble or refractory metal or compound, such as platinum, iridium, tantalum, titanium, titanium nitride, niobium, or alloys of any of these, in order to avoid corrosion or electrolysis which could damage the surrounding tissues and/or the device. In certain embodiments, stimulator 160 is programmable to produce monopolar electrical stimulation, e.g., using the stimulator case as an indifferent electrode, or bipolar electrical stimulation, e.g., using one of the electrodes of the electrode array as an indifferent electrode. For instance, stimulator 160 may have at least four channels and may drive up to sixteen electrodes or more.

Stimulator 160 (which herein refers to implantable pump stimulators, IPG/pump combination stimulators, and/or alternative devices known in the art) contains, when necessary and/or desired, electrical circuitry 154 (FIG. 3) for receiving data and/or power from outside the body by inductive, radio frequency (RF), or other electromagnetic coupling. In some embodiments, electrical circuitry 154 includes an inductive coil for receiving and transmitting RF data and/or power, an integrated circuit (IC) chip for decoding and storing stimulation parameters and generating stimulation pulses (either intermittent or continuous), and additional discrete components required to complete the circuit functions, e.g. capacitor(s), resistor(s), coil(s), and the like. Circuitry 154 may dictate, for instance, the amplitude and duration of the electrical current pulse, when electrical stimulation is used.

Stimulator 160 also includes, when necessary and/or desired, a programmable memory 164 for storing set(s) of data, stimulation, and/or control parameters. Among other things, memory 164 may allow electrical and/or drug stimulation and/or control parameters to be adjusted to settings that are safe and efficacious with minimal discomfort for each individual. Specific parameters and/or medications may provide therapeutic advantages for various types and degrees of CHF. For instance, some patients may respond favorably to intermittent stimulation, while others may require continuous stimulation for treatment and relief. In some embodiments, electrical and drug stimulation parameters are controlled independently, e.g. continuous drug stimulation and no electrical stimulation. However, in some instances, they may advantageously be coupled, e.g., electrical stimulation may be programmed to occur during drug infusion.

Electrical stimulation may be applied as for cardiac pacing and/or cardiac defibrillation. Such stimulation is commonly performed by implantable devices referred to as cardiac pacemakers and implantable cardiac defibrillators (ICDs), respectively. Modern ICDs perform both the pacing and defibrillating functions. Operation of these devices, including stimulation parameters, are well-known to those skilled in the art. An attractive feature of the invention is that the stimulation parameters are programmable and can be adjusted, as required, until an appropriate and efficacious stimulation regime is achieved. Different parameters may have different effects on different tissue. For example, defibrillator pulse shape, duration, voltage, amplitude to adjust minimally required efficacious pulse, pulse width, A-V delay, amplitude, and the like may be adjusted. Different drugs may also have different effects on different tissue. For example, nitrates are useful for acute treatment of exacerbation of heart failure symptoms, while gene therapy agents typically provide relief from symptoms over a much longer time course. Therefore, stimulation and control parameters may be chosen to target specific neural, muscular, and/or other cell populations and to exclude others, or to increase activity in specific neural, muscular, and/or other cell populations and to decrease activity in others. For example, an antiarrhythmic agent may be infused while defibrillation pulses are applied.

Some embodiments of stimulator 160 also include a power source and/or power storage device 166 (FIG. 3). Possible power options for a stimulation device of the present invention, described in more detail below, include but are not limited to an external power source coupled to the stimulation device (e.g., via an RF link), a self-contained power source utilizing any suitable means of generation or storage of energy (e.g., a primary battery, a replenishable or rechargeable battery such as a lithium ion battery, an electrolytic capacitor, a super- or ultra-capacitor, or the like), and if the self-contained power source is replenishable or rechargeable, means of replenishing or recharging the power source (e.g., an RF link, an optical link, a thermal link, or other energy-coupling link).

In embodiments such as depicted in FIG. 3, stimulator 160 includes a rechargeable battery as a power source/storage device 166. The battery is recharged, as required, from an external battery charging system (EBCS) 192, typically through an inductive link 194. In these embodiments, stimulator 160 includes a processor and other circuitry 154 that allow it to generate electrical/infusion pulses that are applied to a patient 208 through electrodes 172 and/or catheter(s) 180 in accordance with a program and stimulation parameters stored in programmable memory 164. As stated earlier, stimulation pulses of drugs include various types and/or rates of infusion, such as intermittent infusion, infusion at a constant rate, and bolus infusion.

According to certain embodiments of the invention, a stimulator operates independently. According to various embodiments of the invention, a stimulator operates in a coordinated manner with other stimulator(s), other implanted device(s), and/or other device(s) external to the patient's body. For instance, a stimulator may control or operate under the control of another implanted stimulator(s), other implanted device(s), and/or other device(s) external to the patient's body. A stimulator may communicate with other implanted stimulators, other implanted devices, and/or devices external to a patient's body via, e.g., an RF link, an ultrasonic link, a thermal link, and/or an optical link. Specifically, a stimulator may communicate with an external remote control (e.g., patient and/or clinician programmer) that is capable of sending commands and/or data to a stimulator and that may also be capable of receiving commands and/or data from a stimulator.

For example, in some embodiments such as shown in FIG. 3, stimulator 160 of the present invention may be activated and deactivated, programmed and tested through a hand held programmer (HHP) 200 (which may also be referred to as a patient programmer and may be, but is not necessarily, hand held), a clinician programming system (CPS) 202 (which may also be hand held), and/or a manufacturing and diagnostic system (MDS) 204 (which may also be hand held). HHP 200 may be coupled to stimulator 160 via an RF link 195. Similarly, MDS 204 may be coupled to stimulator 160 via another RF link 196. In a like manner, CPS 202 may be coupled to HHP 200 via an infra-red link 197; and MDS 204 may be coupled to HHP 200 via another infra-red link 198. Other types of telecommunicative links, other than RF or infra-red may also be used for this purpose. Through these links, CPS 202, for example, may be coupled through HHP 200 to stimulator 160 for programming or diagnostic purposes. MDS 204 may also be coupled to stimulator 160, either directly through RF link 196, or indirectly through IR link 198, HHP 200, and RF link 195.

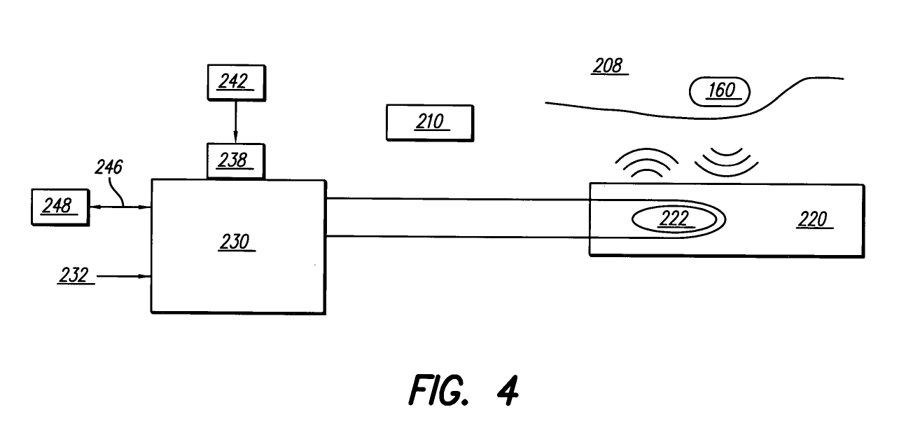

In certain embodiments, and as illustrated in FIG. 4, the patient 208 switches stimulator 160 on and off by use of controller 210, which may be handheld. Stimulator 160 is operated by controller 210 by any of various means, including sensing the proximity of a permanent magnet located in controller 210, sensing RF transmissions from controller 210, or the like.

Additional and alternative exemplary external components for programming and/or providing power to various embodiments of stimulator 160 are also illustrated in FIG. 4. When communication with such a stimulator 160 is desired, patient 208 is positioned on or near external appliance 220, which appliance contains one or more inductive coils 222 or other means of communication (e.g., RF transmitter and receiver). External appliance 220 is connected to or is a part of external circuitry appliance 230 which may receive power 232 from a conventional power source. External appliance 230 contains manual input means 238, e.g., a keypad, whereby the patient 208 or a caregiver 242 may request changes in electrical and/or drug stimulation parameters produced during the normal operation of stimulator 160. In these embodiments, manual input means 238 include various electromechanical switches and/or visual display devices that provide the patient and/or caregiver with information about the status and prior programming of stimulator 160.

Alternatively or additionally, electronic appliance 230 is provided with an electronic interface means 246 for interacting with other computing means 248, such as by a serial interface cable or infrared link to a personal computer or to a telephone modem or the like. Such interface means 246 may permit a clinician to monitor the status of the implant and prescribe new stimulation parameters from a remote location.

One or more of the external appliance(s) may be embedded in a cushion, mattress cover, garment, or the like. Other possibilities exist, including a strap, patch, or other structure(s) that may be affixed to the patient's body or clothing. External appliances may include a package that can be, e.g., worn on the belt, may include an extension to a transmission coil affixed, e.g., with a VELCRO® band or an adhesive, or may be combinations of these or other structures able to perform the functions described herein.

In order to help determine the amount and/or type(s) of stimulating drug(s), and optionally, the strength and/or duration of electrical stimulation, required to produce the desired therapeutic effect, in some embodiments, a patient's response to and/or need for treatment is sensed. A stimulator may incorporate means of sensing CHF (for example, by sensing transmyocardial impedance, intra-ventricular impedance, evoked response latency or duration, ejection fraction, and/or end diastolic pressure), cardiac output (for example, via dilution methods such as dye dilution or thermal dilution, or by sensing ejection fraction by intra-ventricular impedance), or CHF symptoms, e.g., low hemoglobin oxygen saturation or low cardiac output (via bioimpedance or other type(s) of impedance measurement, for instance), or other measures of the state of the patient, such as medication levels, hormone levels, or levels of other blood-borne compounds such as pro-atrial natriuretic peptide (pro-ANP) or brain natriuretic peptide (BNP). For instance, for thermal dilution measurements, an electrically resistive heating element can heat the blood while a thermistor or thermocouple senses the temperature. Alternatively, water (e.g., cold or room temperature water) can be introduced and the temperature change measured. In another example, the electrical activity produced in response to stimulation may be detected, e.g., via recording of the associated electrocardiograph (ECG). When catheters and/or electrodes of a stimulator are implanted, for example, in and/or near the left coronary artery or its branches, signals from an ECG sensor built into the stimulator may be used to adjust stimulation parameters. (As used herein, “adjacent” or “near” means as close as reasonably possible to target tissue(s), including touching or even being positioned within the tissue, but in general, may be as far as can be reached with the stimulation pulses).

Alternatively, a “stimulator” dedicated to sensory processes communicates with a stimulator that provides the electrical and/or infusion pulses. For instance, a microstimulator, such as a BION® manufactured by Advanced Bionics of Sylmar, Calif., may be used to detect abnormal cardiac electrocardiogram (ECG) events. For instance, a BION may use one of many algorithms for analyzing ECGs. These algorithms can operating in the frequency domain, time domain or both. They may employ linear, non-linear, or statistical analysis to categorize the electrogram as originating from various modes, i.e., normal sinus rhythms, sinus tachycardia, ventricular tachycardia, and ventricular fibrillation. In addition, by finding the p, R, and T waves or analyzing the timing of the relationships and durations of the p-wave, QRS complex, and T-wave, it is possible to identify various disease states, such as arrhythmias (irregular heartbeats), hypertrophy (enlarged heart), and tachycardia (accelerated heartbeat), and make predictive diagnosis about perfusion and function of the myocardium. Depending on the extent of CHF, patients may have complications detectable by ECG, including arrhythmias, hypertrophy, and tachycardia. See, for instance, U.S. Pat. No. 5,513,644, titled “Cardiac arrhythmia detection system for an implantable stimulation device,” which is incorporated herein by reference in its entirety.

In another example, the microstimulator could contain one or more accelerometers for detecting the motion of the myocardium. From analysis of the acceleration of the myocardium the contractility of the myocites may be inferred. Poor or decreasing contractility may be an indicator of CHF. See, for instance, U.S. Pat. No. 5,549,650, titled “System and method for providing hemodynamically optimal pacing therapy,” which is incorporated herein by reference in its entirety.

As described below, implant circuitry 154 may, if necessary, amplify and transmit these sensed signals, which may be analog or digital. A stimulator may incorporate other means of sensing CHF or symptoms thereof, in order to determine the required stimulation, including sensing cardiac output, blood pressure, impedance, muscle activity (e.g., EMG), nerve activity (e.g., ENG), observing the stimulation required to decrease or eliminate pain, and/or sensing levels or changes of any blood borne substance, including medications, hormones, neurotransmitters and/or their associated breakdown products, interleukins, cytokines, lymphokines, chemokines, growth factors, enzymes, or other substances, such as CK-MB, ketones, electrolytes, blood glucose concentration, and/or other methods mentioned herein, and yet others evident to those of skill in the art upon review of the present disclosure. For instance, one or more Chemically Sensitive Field-Effect Transistors (CHEMFETs), such as Enzyme-Selective Field-Effect Transistors (ENFETs) or Ion-Sensitive Field-Effect Transistors (ISFETs, as are available from Sentron CMT of Enschede, The Netherlands), may be used. The sensed information may be used to control stimulation parameters in a closed-loop manner.

Therefore, in several embodiments of the present invention, a first and second “stimulator” are provided. The second “stimulator” periodically (e.g. once per minute) records end diastolic pressure and transmits it to the first stimulator. The first stimulator uses the sensed information to adjust drug and/or electrical stimulation parameters according to an algorithm programmed, e.g., by a clinician. For example, the infusion rate of a stimulating drug, such as nitroglycerin, a beta blocker, or fosinopril, may be increased in response to increased end diastolic pressure. In some alternatives, one stimulator performs both the sensing and stimulating functions, as discussed in more detail presently.

While a stimulator may also incorporate means of sensing CHF or symptoms thereof, as described above, it may alternatively or additionally be desirable to use a separate or specialized implantable device to record and telemeter physiological conditions/responses in order to adjust electrical stimulation and/or drug infusion parameters. This information may be transmitted to an external device, such as external appliance 220, or may be transmitted directly to implanted stimulator(s) 160. However, in some cases, it may not be necessary or desirable to include a sensing function or device, in which case stimulation parameters are determined and refined, for instance, by patient feedback, or the like.

Thus, it is seen that in accordance with the present invention, one or more external appliances may be provided to interact with stimulator(s) 160, and may be used to accomplish, potentially among other things, one or more of the following functions:

Function 1: If necessary, transmit electrical power from the external electronic appliance 230 via appliance 220 to stimulator 160 in order to power the device and/or recharge the power source/storage device 166. External electronic appliance 230 may include an automatic algorithm that adjusts drug and/or electrical stimulation parameters automatically whenever the stimulator(s) 160 is/are recharged.

Function 2: Transmit data from the external appliance 230 via the external appliance 220 to stimulator 160 in order to change the parameters of drug and/or electrical stimulation used by stimulator 160.

Function 3: Transmit sensed data indicating a need for treatment or in response to stimulation from stimulator 160 to external appliance 230 via external appliance 220.

Function 4: Transmit data indicating state of the stimulator 160 (e.g., battery level, drug level, electrical stimulation and/or infusion settings, etc.) to external appliance 230 via external appliance 220.

By way of example, referring for example to FIG. 4, a treatment modality for CHF may be carried out according to the following sequence of procedures:

1. A stimulator 160 is implanted so that its catheter discharge portion 182 and electrodes 172 are located at the right atrial appendage and/or RV apex. If necessary or desired, additional leads 170′ and/or catheters 180′ may be used so that, for example, electrodes 172′ and/or catheter discharge portions(s) 182′ may additionally or alternatively be located in or near the coronary sinus and/or great vein.

2. Using Function 2 described above (i.e., transmitting data) of external electronic appliance 230 and external appliance 220, the stimulator 160 is commanded to infuse amounts of an ACE inhibitor, e.g., fosinopril, or a cardiac gene therapy agent, e.g., a vector to deliver the SERCA2a gene, such as an edenovirus carrying the SERCA2a gene, possibly while producing a series of electrical stimulation pulses.

3. After each electrical/infusion pulse, series of stimulation pulses, or at some other predefined interval, any change in, e.g., ejection faction measured by impedance resulting from the stimulation is sensed, for instance, by one or more electrodes 172 and/or 172′ acting as sensors. If necessary, these responses are converted to data and telemetered out to external electronic appliance 230 via Function 3.

4. From the response data received at external appliance 230, or from other assessment, the stimulus threshold for obtaining a response is determined and is used by a clinician 242 acting directly 238 or by other computing means 248 to transmit the desired electrical and/or drug stimulation parameters to stimulator 160 in accordance with Function 2. Alternatively, external appliance 230 makes the proper adjustments automatically, and transmits the proper stimulation parameters to stimulator 160. In yet another alternative, stimulator 160 adjusts stimulation parameters automatically based on the sensed response.

5. When patient 208 desires to invoke electrical stimulation and/or drug infusion, patient 208 employs controller 210 to set stimulator 160 in a state where it delivers a prescribed stimulation pattern from a predetermined range of allowable stimulation patterns.

6. Patient 208 employs controller 210 to turn off stimulator 160, if desired.

7. Periodically, the patient or caregiver recharges the power source/storage device 166 of stimulator 160, if necessary, in accordance with Function 1 described above (i.e., transmit electrical power).

In another example, referring now to FIG. 3, a treatment modality for CHF may be carried out according to the following sequence of procedures:

1. Stimulator 160 is implanted in the thorax, abdomen, or other remote location, and its catheter 180 and possibly also lead 170 tunneled so that its catheter discharge portion 182 and possibly also electrodes 172 are located at the right ventricle. If necessary or desired, additional catheters 180′ and/or leads 170′ may be used so that, for example, catheter discharge portions(s) 182′ and/or electrodes 172′ may additionally or alternatively be located at the coronary sinus and/or great vein.

2. Using HHP 200 described above, stimulator 160 is commanded to infuse a bolus of a nitrate drug, ACE inhibitor, e.g., fosinopril, and/or nitroglycerin, isosorbide dinitrate, or a cardiac gene therapy agent, e.g., a vector to deliver the SERCA2a gene, such as an adenovirus carrying the SERCA2a gene, possibly while producing a series of electrical stimulation pulses.

3. After each electrical/infusion pulse, series of stimulation pulses, or at some other predefined interval, any change in, e.g., ejection fraction measured by impedance resulting from the stimulation is sensed, for instance, by one or more electrodes 172 and/or 172′ acting as sensors. If necessary, these responses are converted to data and telemetered to HHP 200, and from there to CPS 202.

4. From the response data received at CPS 202, the stimulus threshold for obtaining a response is determined and is used by a clinician using CPS 202 and/or HHP 200 to transmit the desired electrical and/or drug stimulation parameters to stimulator 160. Alternatively, the response data are converted, if necessary, and/or used directly by stimulator 160 to modify stimulation parameters in a closed-loop manner.

5. When patient 208 desires to invoke drug and/or electrical stimulation, the patient employs HHP 200 to set stimulator 160 in a state where it delivers a prescribed stimulation pattern from a predetermined range of allowable stimulation patterns.

6. Patient 208 employs HHP 200 to turn off stimulator 160, if desired.

7. Periodically, the patient or caregiver recharges the power source/storage device 166 of stimulator 160, if necessary, using EBCS 192.

For the treatment of any of the various types and severities of CHF, or symptoms thereof, it may be desirable to modify or adjust the algorithmic functions performed by the implanted and/or external components, as well as the surgical approaches, in ways that would be obvious to skilled practitioners of these arts. For example, in some situations, it may be desirable to employ more than one stimulator 160, each of which could be separately controlled by means of a digital address. Multiple channels and/or multiple patterns of drug and/or electrical stimulation might thereby be programmed by the clinician and controlled by the patient in order to, for instance, deal with complex or multiple symptoms or conditions, such as may occur as a result of a change in CHF condition or lead migration, for instance.

In some embodiments discussed earlier, stimulator 160, or two or more stimulators, are controlled via closed-loop operation. A need for and/or response to stimulation is sensed via stimulator 160, or by an another implanted or external device. If necessary, the sensed information is transmitted to stimulator 160. In some cases, the sensing and stimulating are performed by one stimulator. In some embodiments, the parameters used by stimulator 160 are automatically adjusted based on the sensed information. Thus, the drug and/or electrical stimulation parameters may be adjusted in a closed-loop manner to provide stimulation tailored to the need for and/or response to the electrical and/or drug stimulation.

As another example, a first stimulator 160, implanted beneath the skin of the patient 208, may provide a first medication or substance; a second stimulator 160′ may provide a second medication or substance; and a third stimulator 160″ may provide electrical stimulation via electrodes 172 and 172′. As mentioned earlier, the implanted devices may operate independently or may operate in a coordinated manner with other similar implanted devices, other implanted devices, or other devices external to the patient's body. That is, in accordance with certain embodiments of the invention, an external device, e.g., HHP 200, CPS 202, MDS 204, controller 210, computing means 248, and/or the like, controls the operation of one or more of the implanted devices 160, 160′ and 160″. According to various embodiments of the invention, an implanted device, e.g., stimulator 160, may control or operate under the control of another implanted device(s), e.g., stimulator 160′ and/or stimulator 160″. That is, a device made in accordance with the invention may communicate with other implanted stimulators, other implanted devices, and/or devices external to a patient's body, e.g., via an RF link, an ultrasonic link, a thermal link, an optical link, or the like. Specifically, stimulator 160, 160′, and/or 160″, made in accordance with the invention, may communicate with an external remote control (e.g.,HHP 200, CPS 202, MDS 204, computing means 248, controller 210, and/or the like) that is capable of sending commands and/or data to implanted devices and that may also be capable of receiving commands and/or data from implanted devices.

A drug infusion stimulator made in accordance with the invention may incorporate communication means for communicating with one or more external or site-specific drug delivery devices, and, further, may have the control flexibility to synchronize and control the duration of drug delivery. The associated drug delivery device typically provides a feedback signal that lets the control device know it has received and understood commands. The communication signal between the implanted stimulator and the drug delivery device may be encoded to prevent the accidental or inadvertent delivery of drugs by other signals.

Various embodiments of the present invention use one or more drugs to treat CHF acutely. According to such embodiments, one or more of the infused drugs is a medication used for acute treatment of CHF, such as nitroglycerin, nitroprusside, nitric oxide, a nitric oxide donor, adenosine, a loop diuretic (e.g., furosemide), and/or a vasopressin antagonist. Such acute medication may be delivered on demand when the patient indicates such delivery is required, such as via depression of an implanted button or via a remote control that is in communication with the stimulator. The dosage may also be programmed by a clinician, as described earlier. If the stimulator has sensing capability, also discussed earlier, such acute medication may alternatively be delivered on demand when the stimulator senses a change in cardiac function. For example, nitroglycerin might be delivered by the stimulator when it senses low cardiac output.

Certain embodiments of the present invention use one or more drugs to treat CHF chronically. According to such embodiments, one or more of the infused drugs is a medication used for chronic treatment of CHF, such as an ACE inhibitor, an Angiotensin II receptor antagonist, hydralazine, isosorbide dinitrate, isosorbide mononitrate, amyl nitrite, nitric oxide, a nitric oxide donor, a calcium-channel blocker (e.g., amlodipine), digitalis, digoxin, another inotropic agent (e.g., vesnarinone, milrinone, flosequinan, amrinone), a diuretic, a beta-blocker, an antithrombotic agent (e.g., warfarin), an antiarrhythmic agent, ipratropium, theophylline, a vasopressin antagonist, an agent to block TNF production, a TNF antagonist, atrial natriuretic peptide (ANP), brain natriuretic peptide (BNP), a neutral endopeptidase (NEP) antagonist, an antihyperlipidemic agent (e.g., niacin, lovastatin), an endothelin receptor antagonist (e.g., bosentan), and/or an aldosterone antagonist. Such chronic medication may be delivered at a basal rate or via periodic bolus, as programmed by a clinician. The dosage may also be programmed with other drug delivery algorithms by a clinician. Once again, sensing capabilities described earlier may be used for adjustments to chronic treatment.

Various embodiments of the present invention use one or more drugs that improve myocyte calcium handling to treat CHF. According to such embodiments, one or more of the infused drugs is a medication, protein, gene therapy agent, or other agent used to increase the activity of sarcoplasmic reticulum Ca(2+) ATPase (SERCA2a) activity, such as an adenovirus carrying a SERCA2a gene and/or an adeno associated virus carrying a phospholamban gene. Such an agent(s) to promote activity of SERCA2a may be delivered at a basal rate or via periodic bolus, as programmed by a clinician. The dosage or amount delivered may also be programmed with other drug delivery algorithms by a clinician. Once again, sensing capabilities described earlier may be used for adjustments to these treatments.

Some forms of the present invention use more than one, even all, of the approaches mentioned above. As such, some combination of drug(s) to treat CHF acutely, drug(s) to treat CHF chronically, and/or SERCA2a activity promoters may provide the best treatment to some patients. Once again, sensing capabilities described earlier may be used for adjustments to and timing of these treatments.

The drugs and other substances described above may be delivered via approaches, systems, and methods described earlier to one or more of the coronary arteries 100 (which herein describes also branches of the coronary arteries), one or more of the coronary veins 102 (also including branches), the aorta 104, the left ventricle 108, the right ventricle 110, the left atrium 112, the right atrium 114, one or more of the pulmonary veins 116, and one or more of the pulmonary arteries 118. As discussed earlier, electrical stimulation may also be applied during infusion of one or more stimulating drugs.

Furthermore, sensing means described earlier may be used to coordinate the subacute and/or chronic treatment of CHF and related morbidities by infusion and optional electrical stimulation, and then, when appropriate, the acute treatment of CHF symptoms. Alternatively, this coordination may be programmed, and not based on a sensed condition. In yet another alternative, this coordination may be controlled by the patient via the patient programmer.

While the invention herein disclosed has been described by means of specific embodiments and applications thereof, numerous modifications and variations could be made thereto by those skilled in the art without departing from the scope of the invention set forth in the claims.

What is claimed is:

1. A method for treating congestive heart failure, comprising:
providing a first stimulator that generates an infusion pulse in accordance with prescribed parameters;
providing a catheter connected to the first stimulator, which catheter includes a discharge portion;
providing a second stimulator that generates an electrical pulse in accordance with prescribed parameters;
providing a lead connected to the second stimulator, which lead includes at least one electrode;
implanting the catheter discharge portion and the at least one electrode near at least one cardiac tissue to be stimulated;
implanting the first stimulator and the second stimulator at a location remote from the at least one tissue to be stimulated;
tunneling the catheter subcutaneously to the first stimulator location;
delivering via the first stimulator and catheter, infusion pulses of at least one drug as at least one treatment for congestive heart failure to the at least one cardiac tissue, which tissue comprises at least one of a coronary artery, a branch of a coronary artery, a coronary vein, a branch of a coronary vein, the aorta, the left ventricle, the right ventricle, the left atrium, the right atrium, a pulmonary vein, and a pulmonary artery; and
delivering via the second stimulator and lead, at least one electrical stimulating pulse to tissue surrounding the at least one electrode.

2. The method of claim 1 where the first stimulator and the second stimulator are one stimulator.

3. The method of claim 1 wherein the at least one electrical stimulating pulse is a defibrillation pulse.

4. The method of claim 3 further comprising infusing an antiarrhythmic agent.

5. The method of claim 1 wherein the drug comprises at least one of nitroglycerin, nitroprusside, nitric oxide, a nitric oxide donor, adenosine, a loop diuretic, a vasopressin antagonist, an ACE inhibitor, and Angiotensin II receptor antagonist, hydralazine, isosorbide dinitrate, isosorbide mononitrate, amyl nitrite, a calcium-channel blocker, digitalis, digoxin, an inotropic agent, a diuretic, a beta-blocker, an antithrombotic agent, an antiarrhythmic agent, an antihyperlipidemic agent, ipratropium, theophylline, a vasopressin antagonist, an agent to block TNF production, a TNF antagonist, atrial natriuretic peptide (ANP), brain natriuretic peptide (BNP), a neutral endopeptidase (NEP) antagonist, an endothelin receptor antagonist, niacin, prostacyclin, an aldosterone antagonist, an agent promoting SERCA2a activity, an adeno associated virus carrying a phospholamban gene, and an adenovirus carrying a SERCA2a gene.

6. The method according to claim 1 wherein the at least one drug provides acute treatment on demand with at least one of nitroglycerin, nitroprusside, nitric oxide, a nitric oxide donor, adenosine, a loop diuretic, an antiarrhythmic agent, an antithrombotic agent, and a vasopressin antagonist.

7. The method according to claim 1 wherein the at least one drug provides chronic treatment delivered at a basal rate or via periodic bolus of at least one of an ACE inhibitor, and Angiotensin II receptor antagonist, hydralazine, isosorbide dinitrate, isosorbide mononitrate, amyl nitrite, nitric oxide, a nitric oxide donor, a calcium-channel blocker, digitalis, digoxin, an inotropic agent, a diuretic, a beta-blocker, an antithrombotic agent, an antiarrhythmic agent, an antihyperlipidemic agent, ipratropium, theophylline, a vasopressin antagonist, an agent to block TNF production, a TNF antagonist, atrial natriuretic peptide (ANP), brain natriuretic peptide (BNP), a neutral endopeptidase (NEP) antagonist, an endothelin receptor antagonist, niacin, prostacyclin, and an aldosterone antagonist.

8. The method according to claim 1 wherein the at least one drug improves myocyte calcium handling.

9. The method according to claim 8 wherein the drug is at least one agent that increases the activity of sarcoplasmic reticulum Ca(2+) ATPase (SERCA2a) activity.

10. The method according to claim 9 wherein the drug is at least one of an adenovirus carrying a SERCA2a gene and an adeno associated virus carrying a phospholamban gene.

11. The method of claim 1 further comprising providing at least one sensor to sense a physical condition, and adjusting the parameters based on the sensed condition.

12. A method for treating chronic heart failure (CHF), comprising:
implanting a fully implantable stimulator system for generating stimulating pulses;
administering via the stimulator system stimulating pulses of at least one acute CHF drug;
administering via the stimulator system stimulating pulses of at least one chronic CHF drug; and
administering via the stimulator system stimulating pulses of at least one drug that reverses CHF.

13. The method of claim 12 wherein the at least one drug that reverses CHF comprises at least one of a drug that improves myocyte calcium handling, an agent that increases the activity of sarcoplasmic reticulum Ca(2+) ATPase (SERCA2a) activity, an adeno associated virus carrying a phospholamban gene, and an adenovirus carrying a SERCA2a gene.

14. The method of claim 12 wherein the at least one drug provides acute treatment on demand with at least one of nitroglycerin, nitroprusside, nitric oxide, a nitric oxide donor, adenosine, a loop diuretic, an antiarrhythmic agent, an antithrombotic agent, and a vasopressin antagonist.

15. The method of claim 12 wherein the at least one drug provides chronic treatment delivered at a basal rate or via periodic bolus of at least one of an ACE inhibitor, and Angiotensin II receptor antagonist, hydralazine, isosorbide dinitrate, isosorbide mononitrate, amyl nitrite, nitric oxide, a nitric oxide donor, a calcium-channel blocker, digitalis, digoxin, an inotropic agent, a diuretic, a beta-blocker, an antithrombotic agent, an antiarrhythmic agent, an antihyperlipidemic agent, ipratropium, theophylline, a vasopressin antagonist, an agent to block TNF production, a TNF antagonist, atrial natriuretic peptide (ANP), brain natriuretic peptide (BNP), a neutral endopeptidase (NEP) antagonist, an endothelin receptor antagonist, niacin, prostacyclin, and an aldosterone antagonist.

16. The method of claim 12 wherein the stimulating pulses are administered to at least one of a coronary artery, a branch of a coronary artery, a coronary vein, a branch of a coronary vein, the aorta, the left ventricle, the right ventricle, the left atrium, the right atrium, a pulmonary vein, and a pulmonary artery.

17. The method of claim 12 further comprising administering electrical stimulation pulses to treat CHF.

18. The method of claim 17 wherein at least one of the electrical stimulation pulses is a defibrillation pulse.
